(19) United States
(12) Patent Application Publication
(10) Pub. No.: US 2016/0073642 A1
(43) Pub. Date: Mar. 17, 2016

(54) PRODUCTION PROCESS FOR BIOMASS AND FENGYCIN METABOLITES OF BACILLUS SPECIES AND COMPOSITIONS THEREOF FOR BIOLOGICAL PEST CONTROL

(71) Applicants: UNIVERSIDAD EAFIT, Medellín (CO); ASOCIACIÓN DE BANANEROS DE COLOMBIA (AUGURA), Medellín (CO)

(72) Inventors: Isabel Cristina CEBALLOS ROJAS, Medellín (CO); Valeska VILLEGAS ESCOBAR, Medellín (CO); Sandra MOSQUERA LÓPEZ, Medellín (CO); John Jairo MIRA CASTILLO, Medellín (CO); Jaime Andrés GUTIERREZ MONSALVE, Envigado (CO); Juan José ARROYAVE TORO, Medellín (CO); Luisa Fernanda POSADA URIBE, Medellín (CO)

(73) Assignee: UNIVERSIDAD EAFIT, Medellín (CO)

(21) Appl. No.: 14/888,926

(22) PCT Filed: May 2, 2014
(86) PCT No.: PCT/IB14/61167

§ 371 Date: Nov. 3, 2015

Related U.S. Application Data
(60) Provisional application No. 61/819,258, filed on May 3, 2013.

(51) Int. Cl. A01N 63/02; C12N 1/20

(52) U.S. Cl. CPC A01N 63/02; C12N 1/20

(57) ABSTRACT

The present invention refers to a process for increasing the production of biomass and metabolites of microorganisms of Bacillus sp. species. Obtained metabolites are lipopeptide compounds of the fengycin, surfactin, and iturin families, which exhibit antimicrobial activity.

The invention further includes biocidal compositions comprising Bacillus subtilis EA-CB 0015, Bacillus amyloliquefaciens EA-CB0959, and/or metabolites thereof, either alone or together with other biocidal agents, and the use of these compositions for the treatment of diseases caused by various phytopathogenic agents, including Mycosphaerella fijiensis, in a variety of crops.

### FIELD OF THE INVENTION

The present invention refers to a process for increasing the production of biomass and metabolites of Bacillus species, including Bacillus subtilis and Bacillus amyloliquefaciens. The process includes a suitable culture medium and specific environmental conditions, allowing for the production of large amounts of biomass and metabolites of the fengycin, surfactin, and iturin families, which exhibit antifungal and antibacterial activity against various phytopathogenic agents.

### PRIOR ART

Among the microorganisms for biological control, bacteria of Bacillus sp. genus have received much attention due to the wide variety of antibiotic compounds they produce, their long shelf life, their fast growth in culture, and their ability to colonize leaf surfaces [1, 2, 3, 4]. In particular, certain species of Bacillus such as Bacillus subtilis, Bacillus amyloliquefaciens, Bacillus cereus, Bacillus mycoides, Bacillus anthracis, and Bacillus thurigiensis show antimicrobial activity [4].

The antimicrobial activity of these bacteria is due to their ability to produce lipopeptides of the surfactin, iturin, and fengycin families, which differ in the amino acid sequence and the branching of the fatty acid chain. Surfactins exhibit high antibacterial activity, whereas iturins and fengycins are recognized for their antifungal activity [4].

The prior art describes the use of B. subtilis and B. amyloliquefaciens to control various disease-causing microorganisms in a wide variety of crops, including fruit and vegetable crops such as blackberry, grape, raspberry, strawberry, tomato, cucumber, black pepper, orange, melon, apple, peach, custard apple, banana, papaya, mango, and kiwi.

EP2311936 discloses a B. subtilis strain KS1 (NITE BP-569) as a biological control agent to counteract several phytopathogenic microorganisms in vine crops. WO 98/21968 discloses an antibiotic produced by B. subtilis AQ153 (ATCC 55614) effective against bacterial and fungal infections and also as method for protecting plants that comprises the application of these antibiotic compounds.

WO9850422 and WO0029426 disclose other antibiotic compounds produced by the B. subtilis strain AQ713 (NRRL B21661) and its mutants, which exhibit insecticidal, antifungal, and antibacterial activity. WO9909819 discloses antibiotics of a B. subtilis strain AQ 713 (NRRL No. 21665), which produces metabolites with pesticidal activity and a high-molecular-weight metabolite, soluble in water, which exhibits insecticidal and nematicidal activity against corn rootworm and other nematodes.

US2011/0318386 describes methods for inducing systemic resistance against various pathogens through the use of biological controllers of the Bacillus genus, specifically of the isolated B. mojavensis 203-7 and isolated B. mycoides species. In turn, ES 2345969 describes a phytostrengthener for application on banana and plantain pseudostems, which includes B. subtilis, Trichoderma viride, and B. megaterium var phosphaticum.

US2012/00031999 discloses a control strategy for various fungal diseases, including Black Sigatoka in banana, based on the application of synthetic fungicides with some biocontrol microorganisms and their metabolites (specifically B. subtilis strain QST 713, corresponding to the strain of the commercial product Serenade®).

In the production processes of microorganisms of Bacillus species disclosed in the prior art, the amount of biomass produced is very low, given the obtained cell density is not generally greater than 5.0 g/L [8, 9]. Thus, it is necessary to develop new processes to increase the production of biomass and its active metabolites.

Similarly, it is necessary to develop biocide compositions from these microorganisms and/or their biologically active metabolites with a greater efficiency and selectivity for controlling different phytopathogenic agents on a variety of crops.

### BRIEF DESCRIPTION OF THE DRAWINGS

FIG. 1 Structure of the fengycins C produced by Bacillus subtilis EA-CB0015

FIG. 2 Cell density of Bacillus subtilis EA-CB0015 in different culture media.

FIG. 3 HPLC chromatogram of the compounds of Bacillus subtilis EA-CB0015

FIG. 4 MS/MS spectrum of the P5 purified lipopeptide (m/z 1429.9) of Bacillus subtilis EA-CB0015.

FIG. 5 MS/MS spectrum of the P5 purified lipopeptide (m/z 1447.8) after alkaline hydrolysis of the lactone ring of Bacillus subtilis EA-CB0015.

FIG. 6 Area under the curve of the lipopeptides produced by Bacillus subtilis EA-CB0015 in various culture media.

FIG. 7 Percentages of inhibition generated by Bacillus subtilis EA-CB0015 on various phytopathogenic microorganisms.

FIG. 8 Growth inhibition percentages of Mycosphaerella fijiensis generated by Bacillus subtilis EA-CB0015 CFS obtained in various culture media.

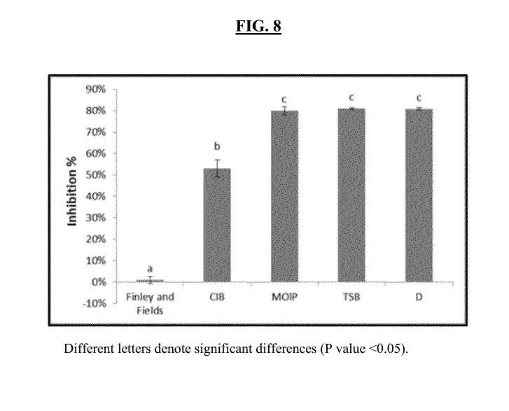

FIG. 9 Viability of Bacillus subtilis EA-CB0015 contained in various compositions of the invention.

FIG. 10 Degree of severity of Black Sigatoka in banana plants treated with different compositions of the invention at a greenhouse level.

FIG. 11 Percentage of necrotic area caused by Black Sigatoka in banana leaves treated with different compositions of the invention.

FIG. 12 Effect of various adjuvant UV protectors on the viability of Bacillus subtilis EA-CB0015.

FIG. 13 Effect of various adjuvants on the adhesion of the compositions (MH2O and P2) based on Bacillus subtilis EA-CB0015.

FIG. 14 Effect of the water-based mixture composition based on Bacillus subtilis EA-CB0015 on the percentage of necrotic area of banana leaves at greenhouse level.

FIG. 15 Photographs of banana leaves affected by Black Sigatoka disease after treatment with various products.

FIG. 16 Effect of water-based composition of Bacillus subtilis EA-CB0015 on the severity of Black Sigatoka in banana at field level.

FIG. 17 Effect of Bacillus subtilis EA-CB0015 on the severity of the disease caused by Botrytis cinerea EAHP-009 in pompons.

FIG. 18 Effect of Bacillus subtilis EA-CB0015 on Colletotrichum sp. in tree tomato (Cyphomandra betacea).

### BRIEF DESCRIPTION OF THE INVENTION

The present invention allows solving these and other disadvantages as it comprises a process for increasing the production of biomass of microorganisms of the Bacillus genus, including Bacillus subtilis EA-CB0015 and Bacillus amyloliquefaciens EA-CB0959, as well as their biologically active metabolites, such as lipopeptides of the surfactin, iturin, and fengycin families.

Additionally, the present invention includes agrochemical compositions that comprise microorganisms of different Bacillus species, including Bacillus subtilis EA-CB0015, Bacillus amyloliquefaciens EA-CB0959 and/or active metabolites thereof, either alone or in combination with biocidal agents for the control of phytopathogenic agents such as Mycosphaerella fijiensis, Fusarium oxysporum, Ralstonia solanacearum, Botrytis cinerea, Colletotrichum sp., Monilia sp. Rhizoctonia solani and Fusarium solani.

The present invention is also directed towards the use of microorganisms of Bacillus species, including B. subtilis EA-CB0015, B. amyloliquefaciens EA-CB0959, and/or active metabolites thereof and agrochemical compositions containing them, for inhibiting the growth of phytopathogenic microorganisms such as Mycosphaerella fijiensis in agricultural crops.

### DETAILED DESCRIPTION OF THE INVENTION

In order to increase the production of biomass of microorganisms of Bacillus sp. and their metabolites (lipopeptides), the microorganism must be cultured in a suitable medium and under the environmental conditions necessary to increase the production of biomass and its metabolites. To do this, a culture medium is prepared (hereinafter medium D) comprising one or more components selected from the group consisting of carbohydrates, yeast extract, ammonium sulfate, peptone, salts containing magnesium, sulfur, manganese, chlorine, potassium, phosphorus, calcium, and sodium either in a solid, semisolid, or liquid matrix.

Environmental conditions necessary for carrying out the process of the invention include temperature, pH, stirring speed, fermentation time, and aeration. The process of the present invention can be performed on a small scale in a laboratory or at large scale in a bioreactor.

In a preferred embodiment, medium D comprises, in w/v percentages, between 3.2% and 3.4% glucose, between 3.1% and 3.3% yeast extract, between 2.5×10⁻³% and 4.5×10⁻³% manganese sulfate, between 2×10⁻³% and 4×10⁻³% calcium chloride, between 0.08% and 0.12% ammonium sulfate, between 0.35% and 0.45% magnesium sulfate, between 0.04% and 0.12% disodium phosphate and between 0.04% y 0.12% monosodium potassium phosphate.

In another even more preferred embodiment of the invention, culture medium D comprises, in w/v percentages, 3.34% glucose, 3.25% yeast extract, 4.2×10⁻³% manganese sulfate, 3.1×10⁻³% calcium chloride, 0.1% ammonium sulfate, 0.4% magnesium sulfate, 0.05% disodium potassium phosphate, 0.05% monosodium potassium phosphate.

In a preferred embodiment of the process of the invention, the microorganism is incubated for a period between 24 and 96 hours with a stirring speed of 400 to 600 rpm, aeration of 1 to 5 vvm, at a temperature between 20° C. and 40° C., and pH between 5.5 and 7.5. Strong acids such as sulfuric acid and/or strong bases such as sodium hydroxide can be used to control and/or adjust the pH. Surfactants and antifoams may also be added to control foam formation.

The process carried out under the above conditions allows increasing the production of biomass and active metabolites of microorganisms of Bacillus sp. The biomass obtained by the process of the present invention can be separated from the culture medium using conventional methods of centrifugation or microfiltration, whereas the active metabolites can be obtained by extraction with solvents, precipitation, adsorption, or chromatography.

In a preferred embodiment of the invention, the amount of biomass of microorganisms of Bacillus sp. obtained can range between 3.0 and 20.0 g/L, preferably between 7.0 and 18.0 g/L, while the concentration of metabolites can range between 200 and 1500 mg/L, preferably between 500 and 1000 mg/L.

In a preferred embodiment, the process of the invention allows increasing the production of biomass and active metabolites of B. subtilis EA-CB0015 and B. amyloliquefaciens EA-CB0959. Identification by 16S rDNA analysis established that B. subtilis EA-CB0015 corresponds to SEQ ID NO: 1 sequence, which is deposited in GenBank under accession number KC006063.

Metabolites of B. subtilis EA-CB0015 and B. amyloliquefaciens EA-CB0959 obtained by the process of the invention include lipopeptides of the surfactins, iturins, and fengycins types. Analysis by mass spectrometry and chromatographic techniques identified a new fengycin isoform produced by B. subtilis EA-CB0015, called fengycins C, whose amino acid sequence is (Glu1-Orn2-Tyr3-THR4-Glu5-Va16-Pro7-Gln8-Thr9-Ile10), which differs from the fengycin B sequence at position 9 and from the fengycin A sequence at positions 6 and 9.

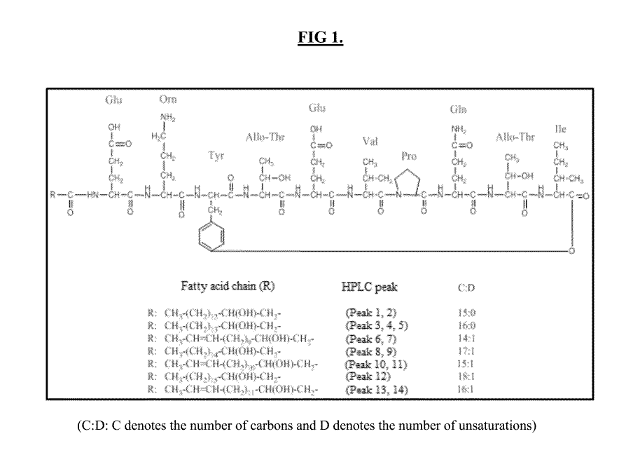

Additionally, following the procedure of the invention for B. subtilis EA-CB0015, this strain can produce 14 different fengycin C homologues with general formula R-Glu1-Orn2-Tyr3-Thr4-Glu5-Va16-Pro7-Gln8-Thr9-Ile10, which vary according to the size of the saturated or unsaturated hydrocarbon chain (R) of 14 to 18 carbons. FIG. 1. illustrates the structures of the various fengycins produced by B. subtilis EA-CB0015 by the process of the invention.

The various homologues of fengycins C, as well as the surfactins and iturins, can be separated by conventional techniques such as high performance liquid chromatography (HPLC). Produced surfactins correspond to different homologues with hydrocarbon chain length between 13 and 16 carbons; iturins correspond to iturins A of 14 and 15 carbons.

In the case of B. amyloliquefaciens EA-CB0959, metabolites produced by the process of the invention correspond to various surfactin homologues (C12 to C15), two iturin A homologues (C14 and C15), and two fengycin isoforms (A and B) with 4 fengycin A homologues (C14, C15, C16, and C17) and 2 fengycin B homologues (C16 and C17).

In a further aspect of the invention, biomass of B. subtilis EA-CB0015 or biomass of B. amyloliquefaciens EA-CB0959 obtained by the process of the present invention inhibits the growth of various plant pathogens such as Mycosphaerella fijensis, Botrytis cinerea, Rhizoctonia solani, Fusarium oxysporum, Fusarium solani, and Colletotrichum sp. This inhibition can be determined using techniques such as dual plates, which involves comparing the growth of plant pathogens when cultured in a medium with and without the presence of the active substances to be assessed. Determined in vitro inhibition percentages are always higher than 50%.

After carrying out the process of the present invention, different compositions or formulations can be prepared from the obtain biomass and/or the metabolites in order to produce physicochemically stable biocidal compositions that ensure the viability of the microorganism and the activity of the metabolites in the composition for long periods.

These compositions can be prepared in a suitable sealed container to avoid contamination. Biomass and/or its metabolites, adjuvants and other ingredients are added to obtain a homogeneous mixture. The final product thus obtained can be collected in suitable containers and stored at room temperature.

In a further aspect, the present invention refers to biocidal compositions comprising B. subtilis EA-CB0015, B. amyloliquefaciens EA-CB0959, and/or their active metabolites, either alone, combined or in association with other active agents to enhance biological activity. The biocidal compositions of the present invention may contain one or more adjuvants and an agrochemically acceptable carrier.

In a preferred embodiment, the biocidal compositions of the present invention comprise between 80.0 and 99.9% w/w of an aqueous suspension of B. subtilis EA-CB0015 at a concentration of 1×10⁷ a 1×10¹¹ CFU/mL together with a mixture composed of 2.0% to 4.0% w/v sodium carboxymethyl cellulose (CMC), 1.0% to 5.0% v/v 3M phosphate buffer (pH 5.0), 1.0% to 4.0% v/v glycerol, 0.25% to 0.75% v/v Tween 20®, 0.25% to 0.5% v/v Triton X-100®, 0.01% to 1.0% v/v potassium sorbate, 0.05% a 0.15% /v xanthan gum, 0.2% to 1.5% w/v skimmed milk, and 0.028% to 1.0% w/v TiO₂. This composition is of white color, has a pH of 4.0 to 6.5, and a viscosity of 20 to 80 cp.

In a further preferred embodiment, the biocidal compositions of the invention also comprise chemical pesticides such as anilinopyrimidines, bitartenols, sterols, difeconazole, tebuconazole, epoxiconazole, mancozeb, chlorothalonil and other agents for the biological control of pests, together with one or more adjuvants in an agrochemically acceptable carrier.

In a further aspect, the present invention is directed to the use of microorganisms of Bacillus sp. particularly of B. subtilis EA-CB0015, B. amyloliquefaciens EA-CB0959 and/or their metabolites, as well as their biocidal compositions, to inhibit the growth of phytopathogenic microorganisms such as Mycosphaerella fijiensis, Fusarium oxysporum, Ralstonia solanacearum, Botrytis cinerea, Colletotrichum sp., Monilia sp. Rhizoctonia solani, and Fusarium solani in agricultural crops.

In a further aspect, the present invention is directed to a method for treating plants against infections caused by various phytopathogens, which comprises applying an effective amount of a microorganism of Bacillus sp. to the plant, particularly B. subtilis EA-CB0015 and B. amyloliquefaciens EA-CB0959 and/or their metabolites, or applying biocide compositions containing them, either alone or in combination with other biocidal agents.

The application can be done by spraying at a dose ranging from 0.1 to 10 liters per hectare (L/ha) in admixture with an appropriate carrier or mixed with other compositions containing one or more pesticides.

The following examples further illustrate the invention, although the inventive concept is not restricted thereto.

### EXAMPLES

### Example 1

### Obtaining Bacillus sp.

Strains of Bacillus sp. were obtained from cv. Gran enano and cv. Valery cultivars, both of bananas, and cv. Harton of plantain. A plantation was selected for each cultivar and five points were established to collect composite samples of three plants before flowering using random probability sampling without replacement. Sampling was performed on leaves number 2, 5, and 10 of each plant and each leave was split to select an area of the apex and an area of the base.

Bacterial isolation was carried out by washing with a sodium phosphate buffer and Tween 80® and performing sonication of the samples. Serial dilutions were made and plated on TSA surface (Trypticase Soy Agar, Merck at 10%). Gram positive cells were purified, cultured in Finley and Field's medium (150 rpm, 4 days, 30° C.) and subjected to heat shock (80° C., 20 min). All AEFBs (Aerobic Endospore-Forming Bacteria) were stored in TSB (Tryptic Soy Broth, Merck) and glycerol (20% v/v) at −80° C., and activated in TSA at 50% prior to any experimental use.

### Example 2

### Obtaining Biomass of Bacillus subtilis EA-CB0015 and Bacillus amyloliquefaciens EA-CB0959

B. subtilis EA-CB0015 strain was replicated in TSA 50% and incubated for 48 hours at 30° C. A colony of the strain was inoculated in culture medium D and incubated for 12 hours at 30° C. and 200 rpm. This culture was used as pre-inoculum. Fermentation was carried out in 50-mL flasks with 10 mL of culture medium D at a temperature of 30° C. and 200 rpm in an orbital shaker. Each Erlenmeyer was inoculated with 1 mL of a bacterial suspension adjusted to an OD₆₀₀ of 1 and obtained after 12 hours of growth. Cell densities of up to 13.2±1.7 g/L of B. subtilis EA-CB0015 were obtained.

In order to assess the performance of the process in obtaining biomass of B. subtilis EA-CB0015, the amount of biomass obtained using the culture medium of the invention (medium D) was compared with the amount of biomass obtained using CIB, MOLP, Finley and Field's, and TSB culture media.

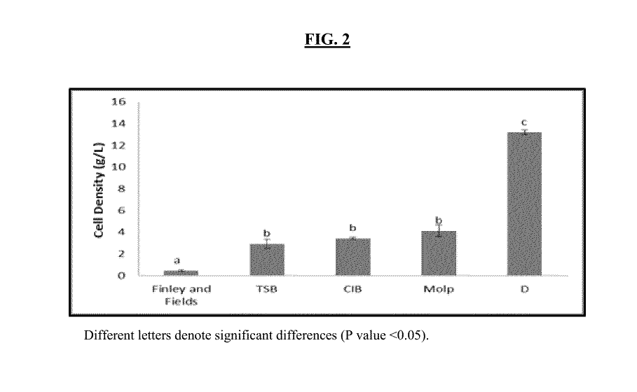

Cell density obtained in the culture medium of the invention was 29.3 times greater than that obtained in Finley and Field's medium (0.6±0.1 g/L), 4.5 times greater than that obtained in TSB medium (2.95±0.4 g/L), 3.6 times greater than that obtained in the CIB medium (3.65±0.8 g/L), and 3.2 times greater than that obtained in the MOLP medium (4.1±0.6 g/L), as illustrated in FIG. 2.

Following the same procedure, biomass of B. amyloliquefaciens EA-CB0959 was obtained. As for B. subtilis EA-CB0015, the amount of biomass obtained using the culture medium of the invention (medium D) was higher than that obtained in the MOLP and TSB media. Cell densities obtained with the medium of the invention range between 8.0 and 10.0 g/L.

### Example 3

### Extracting and Determining Active Metabolites of Bacillus subtilis EA-CB0015 and B. amyloliquefaciens EA-CB0959

From B. subtilis EA-CB0015 culture obtained according to Example 2, an extraction process of their active metabolites was performed with methanol. Subsequently, a solid phase extraction (SPE) was carried out with methanol as the organic solvent and active fractions were purified by reverse phase HPLC with an UV detector at a wavelength of 214 nm.

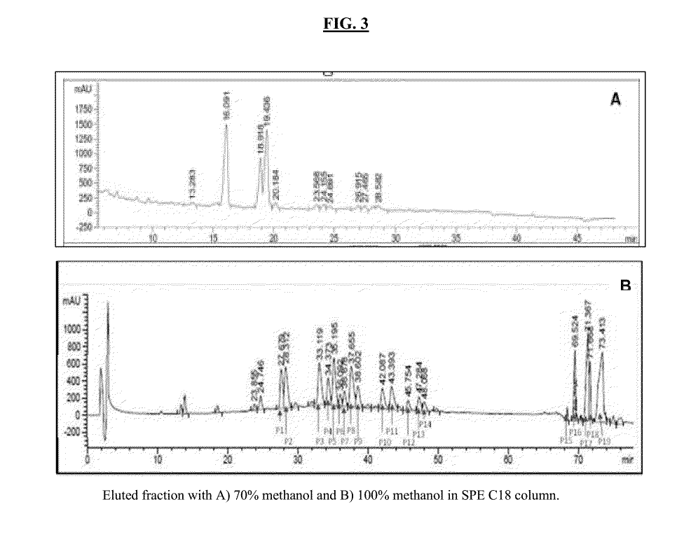

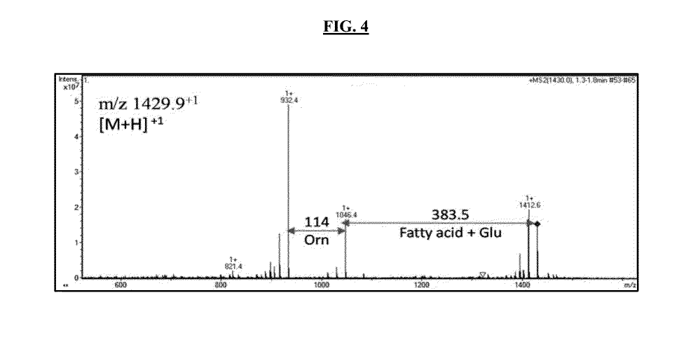

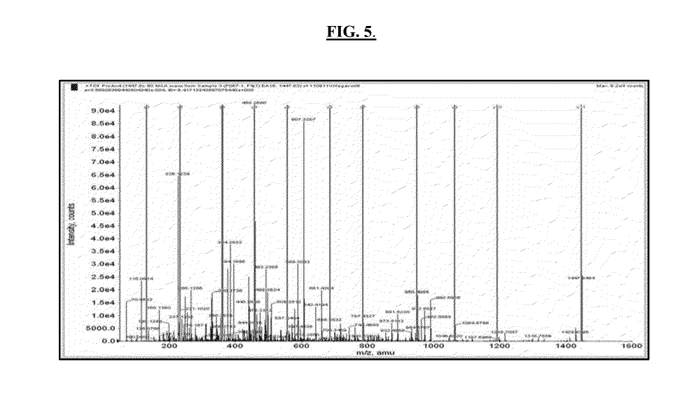

FIG. 3 illustrates the respective chromatograms. Eluted peaks between minute 16 and 19 correspond to iturins A (FIG. 3A), peaks P1 to P14 (FIG. 3B) correspond to fengycins C, and peaks P15 to P19 (FIG. 3B) correspond to surfactins. Some of the active metabolites were also identified by ESI-MS/MS (electrospray mass spectrometry), as shown in FIGS. 4 and 5:

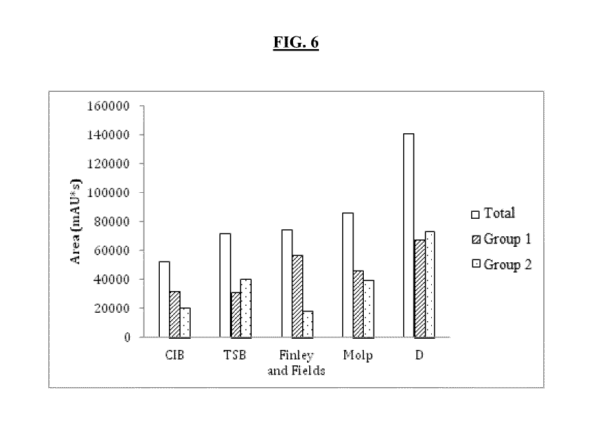

In order to assess the performance of the process in obtaining the two groups of active metabolites of B. subtilis EA-CB0015, the amount of metabolites obtained using the culture medium of the invention (medium D) was compared with the amount of metabolites obtained using CIB, MOLP, Finley and Field's, and TSB culture media. Peak areas and thus the amount of metabolites obtained were greater when using culture medium D of the invention in the process, as shown in FIG. 6.

Following the same extraction and HPLC purification procedure previously mentioned, metabolites produced by B. amyloliquefaciens EA-CB0959 were identified, corresponding to various surfactin homologues (C12 to C15), two iturin A homologues (C14 and C15), and two fengycin isoforms (A and B) with 4 fengycin A homologues (C14, C15, C16, and C17) and 2 fengycin B homologues (C16 and C17).

### Example 4

### Evaluating the Activity of Bacillus subtilis EA-CB0015 and Bacillus amyloliquefaciens EA-CB0959 Against Phytopathogenic Microorganisms

Evaluation of antifungal activity was performed using the ring method. Briefly, a circular print (6 cm of diameter) of B. subtilis EA-CB0015 was made in a Petri dish (9 cm of diameter) with PDA, and then a disk (5 mm of diameter) of the fungus (grown for 10 days) was placed in the center thereof. Petri dishes inoculated only with disks of the fungus were used as absolute control, and the radial mycelial growth was measured when the fungus reached a growth equal to the diameter of the circle formed by the bacteria.

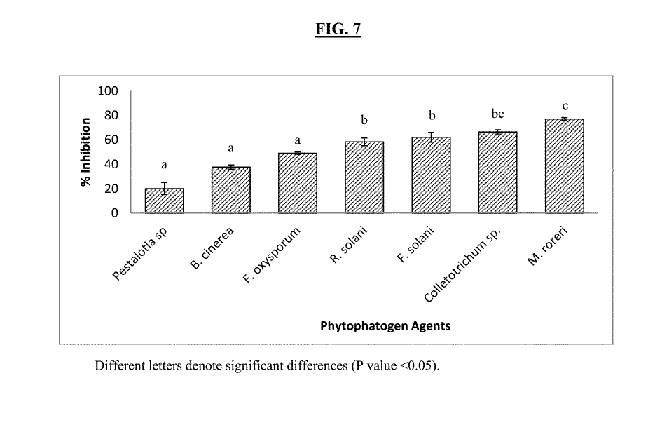

The experiments had a completely randomized univariate design with three replicates per treatment. The established response variable was the percentage of mycelial growth inhibition, which was calculated considering growth of the absolute control as 100%. As FIG. 7 shows, the percentage of inhibition generated by B. subtilis EA-CB0015 was approximately 20% on Pestalotia sp. and 80% on Moniliophthora roreri.

In addition, B. subtilis EA-CB0015 exhibits antibacterial activity against various microorganisms, including Ralstonia solanacearum, generating inhibition zones of up to 6 millimeters in BGTA culture medium. Quantitative antagonism tests against R. solanacearum were performed by surface seeding 100 μL of a R. solanacearum suspension adjusted to 10⁶ CFU/mL in BGTA agar. Then, TSA discs (5 mm) of B. subtilis EA-CB0015 were incubated for 48 hours at 30° C. Finally, the generated inhibition zone was determined after 72 hours.

With the same methodology described above, the activity of B. amyloliquefaciens EA-CB0959 against F. oxysporum, M. fijiensis and R. solanacearum was evaluated, with inhibition percentages of 58.5% and 76.0%, and an inhibition radius of 10.9 mm, respectively.

### Example 5

### Evaluating Bacillus subtilis EA-CB0015 and Bacillus amyloliquefaciens EA-CB0959 Against Mycosphaerella fijiensis

To select the antagonistic bacteria, an initial screening was performed using the microplate technique with the modified methodology of Peláez 2006 [10]. It was quickly established which cell-free supernatants (CFS) of the isolated AEFBs generated mycelial growth inhibition on Mycosphaerella fijiensis when incubated in liquid medium.

For the evaluation, strains of M. fijiensis EASGK09, M. fijiensis EASGK10, M. fijiensis EASGK11, and M. fijiensis EASGK14 fungi were used, isolated from cv. Gran enano banana leaves and following the methodology of Dupont, 1982[11]. CFS of B. subtilis UA321 were used as positive control and fresh sterile broth was used as absolute control.

A dual plates test was conducted with the modified methodology of Riveros [12]. Growth inhibition percentages of fungus colonies were evaluated on PDA (Merck, supplemented with chloramphenicol: 200 ppm and ampicillin: 250 ppm). Inhibition tests were conducted on the germ tube using the modified varnish technique described Talavera [13].

Growth inhibition was evaluated on the germ tube of fungal ascospores discharged on leaf discs of cv. Gran enano banana previously submerged in CFS. The inhibition percentage on the germ tube was determined considering spore germination of the absolute control as 100%.

Bacteria selected as antagonists were those whose CFS showed M. fijiensis growth inhibition percentages higher than those of B. subtilis UA321 positive control. This initial selection process was carried out with 648 AEFBs. AEFBs selected as antagonists of M. fijiensis were tested again using the microplate technique and subjected to dual plates and ascospores inhibition tests using the CFS obtained from fermentation in MOLP culture media [14]. These tests used M. fijiensis EASGK14 strain and the same controls of the initial screening.

Finally, AEFBs selected as antagonists of M. fijiensis were subjected to three additional tests against the fungus: microplates with MOLP culture medium, dual plates, and ascospores inhibition. Then, a weighted average of the three tests was calculated, resulting in values of 60%, 20%, and 20% for the ascospores, dual plates, and microplates tests respectively.

The higher weight associated with the ascospores test relates to the importance of attacking the fungus before it enters leaf stomata. Inhibition percentages of mycelial growth and ascospores germination of M. fijiensis generated by B. subtilis EA-CB0015 and B. amyloliquefaciens EA-CB0959 in vitro are shown in Table 1.

TABLE 1
Inhibition percentages generated by Bacillus sp. against M. fijiensis

| | Mycelial growth | | | |
|---|---|---|---|---|
| | inhibition/Technique: | | | Weighted |
| | | Dual | Inhibition of | average of |
| | Microplates | plates | ascospores | the three |
| Microorganism | in MOLP (%)^{a} | (%)^{b} | growth^{c} | tests^{d} |
| Bacillus subtilis | 89 ± 1 | 78 ± 1 | 98 ± 1 | 92 |
| EA-CB0015 | | | | |
| Bacillus | 89 ± 0 | 62 ± 2 | 77 ± 0 | 76 |
| amyloliquefaciens | | | | |
| EA-CB0959 | | | | |

^{a}Mycelial growth inhibition of M. fijiensis using the microplate technique with MOLP broth; 2 replicates and 4 repetitions per treatment of the experiment performed 2 times over time.
^{b}Mycelial growth inhibition of M. fijiensis using the dual plates in PDA technique; 2 replicates and 30 repetitions per treatment of the experiment performed 2 times over time.
^{c}Germination inhibition of M. fijensis ascospores with two replications for treatment.
^{d}Average of the three antagonism tests: ascospores (60%), microplates (20%), and dual plates (20%).

The growth inhibition percentage of M. fijiensis obtained with B. subtilis EA-CB0015 CFS in medium D was 1.5 higher than that obtained in CIB medium (53.0±4.0%), 80.9 times higher than that obtained in Finley and Field's medium (1.0±1.6%) (FIG. 7). Growth inhibition percentages of M. fijiensis obtained using the CFS in MOLP and TBS media were statistically similar to that obtained in medium D.

In vitro growth inhibition of M. fijiensis by the CFS of the isolated AEFBs suggests they have an impact on the cellular structures of the fungus. For this reason, the presence of morphological changes in the mycelium and ascospores of M. fijiensis was evaluated by light microscopy.

It was observed that the CFS of all antagonist bacteria produced morphological changes, manifested by masses on mycelial hyphae and inhibitions in the germination of the ascospores tube when compared with the absolute control.

### Example 6

### Biocidal Compositions Comprising Bacillus subtilis EA-CB0015

For the development of the biocidal compositions of the invention, a pre-selection of the adjuvants was carried out and the combinations that generate the most stable mixtures and the ratios of each component in the mixture were established by evaluating such aspects as the occurrence of phases and the presence of precipitates. Selected adjuvants and their roles are shown in Table 2.

TABLE 2
Adjuvants of the various compositions.

| Adjuvant^{a} | Product^{b} | Formulation^{c} | Role^{d} |
|---|---|---|---|
| Oil | Sunflower oil | EM; OB | Provides substance to the formulation. |
| | Soybean oil | EM; OB | Preventing microorganism desiccation. |
| | Canola oil | EM; OB | |
| Surfactant | Tween 20 | EM; MH2O | Improving coverage of the hydrophobic |
| | | | surface of the plant. |
| | Tween 80 | EM; MH2O | Helping the mixture of hydrophobic spores |
| | | | with water. |
| | Triton x-100 | EM; MH2O | Helping the mixture of hydrophobic spores |
| | | | with water. |
| Adherent | Xanthan gum | MH2O; EM | Improving adherence of the microorganism to the |
| | | | surface of the leaf. Stabilizing the mixture. |
| Dispersant | Sodium | MH2O | Neutralizing interactions between similar |
| | alginate | | particles to achieve a uniform suspension. Ensuring |
| | Veguum | MH2O | release of the microorganism after application. |
| | Sodium | MH2O | |
| | carboxymethyl | | |
| | cellulose | | |
| | (CMC) | | |
| Humectant | Glycerol | MH2O | Increasing moisture content of the product by absorbing |
| | | | water in the air. Preventing microorganism desiccation. |
| Antimicrobial | Propionic acid | MH2O; EM | Preventing product degradation due to contamination |
| agent | | | |

^{a}Group of adjuvants required for the mixture.
^{b}Adjuvants selected for the evaluation.
^{c}Composition of which they make part. EM: Emulsion, OB: Oil-based composition, MH₂O: Water-based mixture.
^{d}Role of each adjuvant in the mixture.

For the composition in emulsion form, the best combination of surfactant and oil was established using a factorial 3×2 designed (factors: type of oil and type of emulsion, with three levels each) was used. A constant ratio was used to evaluate the components: 20 mL oil, 1 μmol surfactant, and 80 mL water.

After selecting the type of oil and surfactant, a ternary mixture with center point design was carried out in order to determine the ratios of sunflower oil, surfactant, and dispersant (xanthan gum) that should be added to obtain a highly stable emulsion.

Evaluated ranges were 0.3 to 1.0%, 0.0 to 5.0%, and 14.0 to 19.6% for xanthan gum, oil, and surfactant, respectively. Ratios were selected as reported by Burges (1998) and Brar et al (2006) [15, 16]. In addition to the evaluated components, to the mixtures was added a 3M phosphate buffer of pH 5.0 (3%) to offset drastic changes in pH when adding the adjuvants, propionic acid (0.5%) as an antimicrobial agent, and q.s. water 100%.

As for the water-based mixture, the most stable combination of dispersant, surfactant, and adherent was determined using a fractional factorial design that produced eight mixtures. These mixtures were then evaluated in the fractional design to select the water-based mixture adjuvants shown in Table 3. The remainder was completed with water.

TABLE 3
Evaluated mixtures using a fractional design for
selecting water-based mixture adjuvants (MH₂O)

| Mixture | X gum | (CMC) | (veggum) | (alginate) | (tween 20) | (tween 80) | (triton X-100) |
|---|---|---|---|---|---|---|---|
| 1 | − | − | − | + | + | + | − |
| 2 | + | − | − | − | − | + | + |
| 3 | − | + | − | − | + | − | + |
| 4 | + | + | − | + | − | − | − |
| 5 | − | − | + | + | − | − | + |
| 6 | + | − | + | − | + | − | − |
| 7 | − | + | + | − | − | + | − |
| 8 | + | + | + | + | + | + | + |
| −^{a} | 0.1% | 0% | 0% | 0% | 0% | 0% | 0% |
| +^{b} | 0.5% | 1.25% | 1.25% | 1.25% | 2.50% | 2.50% | 2.50% |

^{a}Minimum level for the evaluation of each factor.
^{b}Maximum level for the evaluation of each factor
* The + y − signs denote the level for the evaluation of each factor in the mixture.

Mixture 3 was selected as the most stable and subjected to a ternary mixture with center point design in order to determine the rations of CMC, Tween 20®, and Triton X-100®. Evaluated ranges were 0.5% to 3.5% for the three components tested. These ranges were determined as reported by Burges (1998) and Brar et al (2006) [16, 15].

The concentration of xanthan gum remained constant at the lowest level (0.1%) evaluated in the fractional factorial design since all stable mixtures contained this additive at this concentration. In addition to the evaluated components, to the design mixtures was added a 3M phosphate buffer of pH 5.0 (3%) to offset drastic changes in pH when adding the adjuvants, propionic acid (0.5%) as an antimicrobial agent, and q.s. water 100%.

Mixtures with improved stability over time for the emulsion and the water-based mixture were selected. Water was replaced with bacterial culture B. subtilis EA-CB0015, and its final bacterial concentration was adjusted to 2±×10⁸ CFU/mL.

In addition to the two above mentioned compositions, bacterial suspensions (SB) consisting solely of the bacterial culture obtained after 72 hours of fermentation and conditioned with a 3M phosphate buffer of pH5 (3M K₂HPO₄, 3M KH₂PO₄) (3%) and propionic (0.5%) were evaluated. Table 4 illustrates the compositions of the various formulations evaluated with B. subtilis EA-CB0015.

TABLE 4
Compositions of various formulations based
on Bacillus subtilis EA-CB0015.

| FORMULA- | ACRO- | |
|---|---|---|
| TION | NYM | COMPOSITION |
| Bacterial | BS | Bacterial culture (94.25% v/v-98.75% v/v), |
| suspension | | 3M phosphate buffer of pH 5 (1% v/v-5% v/v), |
| | | and propionic acid (0.25% v/v-0.75% v/v) |
| Composition | MH₂O | Bacterial culture (86.6% v/v-93.2% v/v) |
| water base | | sodium carboxymethyl cellulose (2% w/v-4% |
| | | w/v), 3M phosphate buffer of pH 5 (1% v/v-5% |
| | | v/v), glycerol (1% v/v-4% v/v) Tween |
| | | 20 ® (0.25% v/v-0.75% v/v) Triton X-100 |
| | | (0.25% v/v-0.75% v/v), propionic acid (0.25% |
| | | v/v-0.75% v/v), and xanthan gum (0.05% |
| | | w/v-0.15% w/v). |
| Composition | EM | Bacterial culture (71.4% w/v-83.6% v/v), |
| emulsion base | | sunflower oil (14% v/v-18% v/v), 3M |
| | | phosphate buffer of ph 5 (1% v/v-5% |
| | | v/v), Tween 80 ® (1% v/v-4% v/v), |
| | | xanthan gum (0.2% w/v-0.9% w/v), and |
| | | propionic acid (0.25% v/v-0.75% v/v). |

### Example 7

### Evaluation of the Biocidal Compositions of the Invention

The compositions obtained in Example 6 were used to evaluate their antagonistic capacity against ascospores of M. fijiensis and the viability of B. subtilis EA-CB0015 in the formulation for a given storage time (180 days).

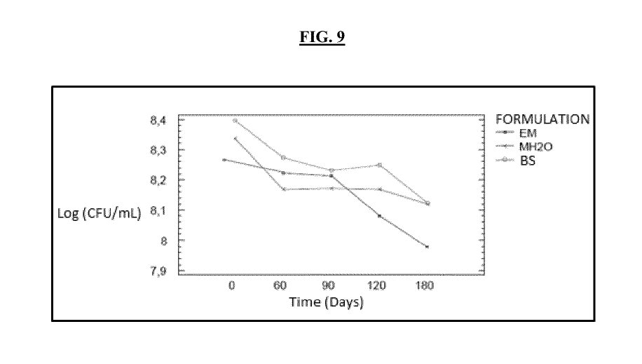

Regarding the evaluation of the viability of the bacteria over time, FIG. 9 shows a gradual decrease in CFU over time for all treatments, with a more marked decrease for the emulsion from the third month. The water-based mixture and the bacterial suspension showed very similar decreases, although the former showed the lowest value.

According to the above, the best compositions in relation to the viability of B. subtilis EA-CB0015 in the formulation during the evaluated time period are those based on water or with a bacterial suspension.

In order to evaluate the effect of the compositions on the development of Black Sigatoka in banana plants, an experiment was conducted to assess the emulsion, the water-based mixture, and the bacterial suspension of B. subtilis EA-CB0015. The compositions were diluted to a concentration of 1.0×10⁸ CFU/mL and applied by spraying 30 drops/cm² on the first leaf completely unfolded after the flag leaf (leaf number one), on which the evaluation was conducted.

Inoculation of the pathogen was performed through artificial inoculation by adding 20 mL of a mycelial suspension of 10-day old M. fijiensis on leaf number one. Inoculation of the pathogen was done 24 hours before applying the compositions of the AEFBs. The degree of severity of the disease was determined 30 days after applying the compositions using the Fouré scale (1982)[17] and the percentage of necrotic leaf area was determined using photos of leaves and the Assess 1.0 image analysis software.

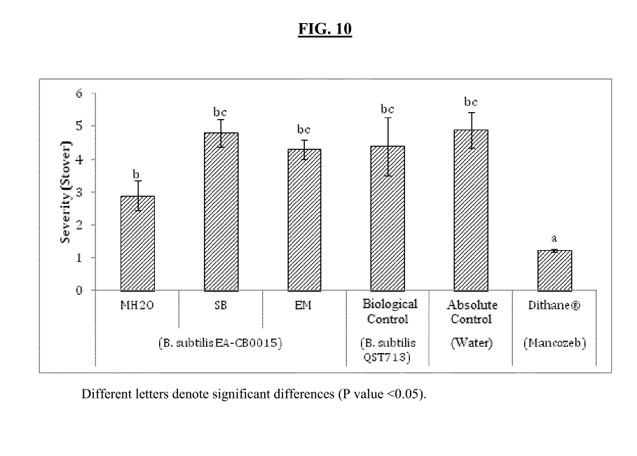

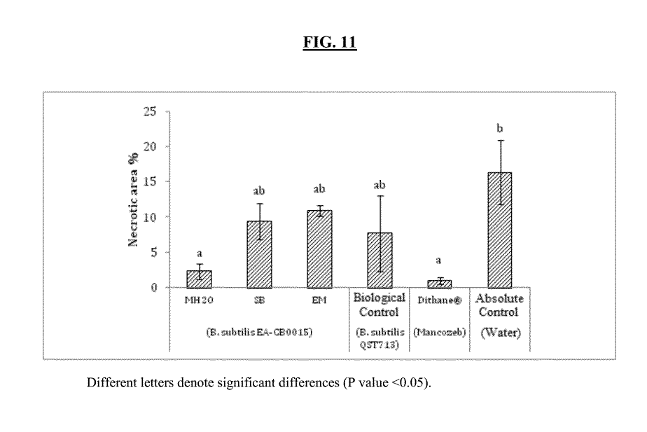

For both measurements, the results of applying sterile water were used as negative control and the data reported for the chemical fungicide Dithane® and the biological fungicide Rhapsody®, employed according to the provider's recommendations, were used as positive control. Results are illustrated in FIGS. 10 and 11, which show the differences in the degree of severity and in the percentage of necrotic area for the various treatments evaluated by analysis of variance (p<0.05).

It is noteworthy that the water-based mixture composition was the only biological treatment that showed significant disease control equal to the chemical control of Dithane® for the two analyzed response variables. This bioformulation reduced the degree of severity to 97.1% and reported a necrotic area of 2.3%, a percentage similar to that obtained by chemical control (1.0%). The degree of severity and the percentage of necrotic area of the negative control was 4.2 and 16.3%, respectively.

Other evaluated treatments showed no disease control, that is, they did not show significant differences when compared with the negative control for any of the analyzed two variables.

### Example 8

### Evaluating the Physicochemical Properties, Adhesion, Resistance to UV Radiation, and Characteristics of the Compositions of the Invention

Adhesion and resistance to UV radiation of the compositions of Example 6 were lower when compared with other chemicals in the market. To improve these properties, an initial selection of adjuvants was carried out. Table 5 shows the adjuvants used to improve adhesion and UV protection, and the range used for their evaluation.

TABLE 5
Adjuvants evaluated to improve adhesion and UV protection
of the compositions of the present invention.

| Role | Adjuvant Type | Concentration (% w/v) |
|---|---|---|
| UV Protector | TiO₂ (Technical) | 0.5-1.5 |
| UV Protector | ZnO₂ (Technical) | 0.5-1.5 |
| UV Protector | Polyvinyl alcohol (PVA) | 0.1-1.0 |
| Adherent | Skimmed milk | 0.1-1.5 |
| Adherent | Pegal ® | 0.01-0.1 |
| Adherent | Sodium caseinate (CaNa) | 0.1-1.0 |

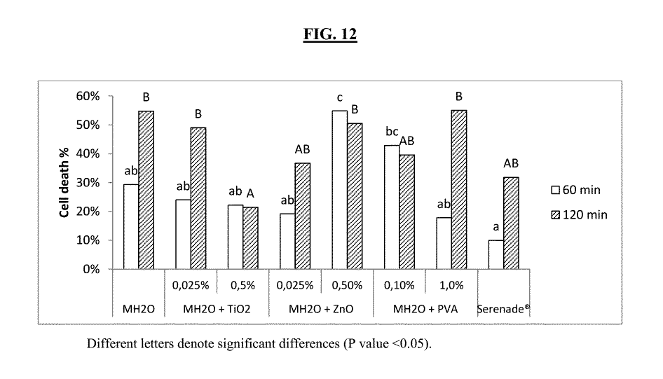

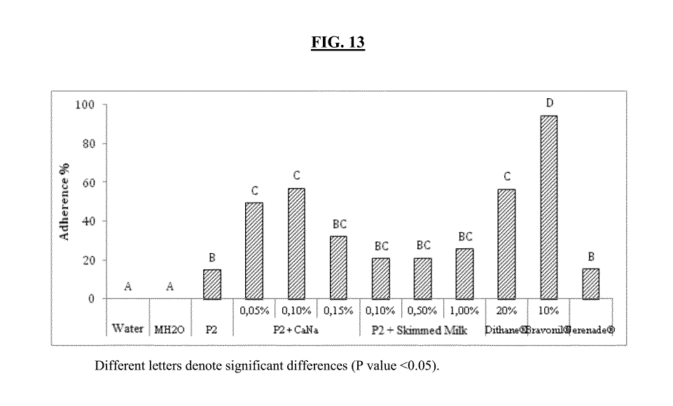

The adjuvants were appraised using cost, market availability, and compatibility with the composition of the invention as criteria. Then, an evaluation of the pre-selected adjuvants was carried out using a multifactorial experimental design where top performers were identified for each evaluation criteria in a specific concentration range (FIGS. 12 and 13).

According to FIG. 12, the water-based bacterial composition (MH₂O) of the invention, supplemented with the adjuvant TiO₂ at 0.5% w/v, improved UV resistance, reducing cell death of B. subtilis EA-CB0015 from 55% to 21.5% after being exposed to UV radiation for 120 minutes. With this finding a new water-based composition was developed, incorporating TiO₂ at 0.5% and replacing propionic acid with potassium sorbate in a composition called P2.

FIG. 13 shows that the addition of sodium caseinate and skimmed milk significantly improved the adhesion of the product to hydrophobic surfaces. Therefore, skimmed milk was added to the water-based composition P2, forming a new composition named P3.

In order to determine whether the composition of B. subtilis EA-CB0015 can act in combination with the chemical fungicide mixtures, the viability of B. subtilis EA-CB0015 was determined before and after subjecting the composition to tank mixtures used for the control of Black Sigatoka in commercial plantations. For this purpose, a sample of 10 mL of the composition was taken and subjected to the various tank mixtures described in Table 6.

TABLE 6
Mixtures of the water-based composition based on Bacillus subtilis
EA-CB005 with various chemical fungicides.

| | Treatment | | | | | | |
|---|---|---|---|---|---|---|---|
| | Water | Agricultural | Dithane ® | Pegal ® | Chlorothalonil ® | Formulation | SICO ® |
| Compound | (% V/V) | Oil (% V/V) | (% V/V) | (% V/V) | (% V/V) | (% V/V) | (% V/V) |
| Mixture 1 | 72.53 | 0 | 0 | 0 | 7.47 | 20 | 0 |
| Mixture 2 | 38.86 | 40.88 | 8.79 | 0.41 | 0 | 9.31 | 1.76 |
| Mixture 3 | 38.51 | 40.88 | 8.79 | 0.41 | 0 | 9.22 | 0 |
| Mixture 4 | 38.51 | 40.88 | 8.79 | 0.41 | 0 | 9.22 | 0 |
| Mixture 5 | 38.86 | 40.88 | 8.79 | 0.41 | 0 | 9.31 | 0 |
| Mixture 6 | 36.75 | 40.88 | 8.79 | 0.41 | 0 | 8.78 | 0 |
| Mixture 7 | 38.86 | 40.88 | 8.79 | 0.41 | 0 | 9.31 | 0 |
| Mixture 8 | 38.51 | 40.88 | 8.79 | 0.41 | 0 | 9.21 | 0 |

| | Treatment | | | | | |
|---|---|---|---|---|---|---|
| | SIGANEX ® | Baycor ® | Impulse ® | OPUS ® | Atlas ® | Calixin ® |
| Compound | (% V/V) | (% V/V) | (% V/V) | (% V/V) | (% V/V) | (% V/V) |
| Mixture 1 | 0 | 0 | 0 | 0 | 0 | 0 |
| Mixture 2 | 0 | 0 | 0 | 0 | 0 | 0 |
| Mixture 3 | 2.2 | 0 | 0 | 0 | 0 | 0 |
| Mixture 4 | 0 | 2.2 | 0 | 0 | 0 | 0 |
| Mixture 5 | 0 | 0 | 1.76 | 0 | 0 | 0 |
| Mixture 6 | 0 | 0 | 0 | 4.4 | 0 | 0 |
| Mixture 7 | 0 | 0 | 0 | 0 | 1.76 | 0 |
| Mixture 8 | | 0 | 0 | 0 | 0 | 2.2 |

For this evaluation, an 8×8 multifactorial design was used, wherein the first factor assessed was the type of tank mixtures with 8 levels, and the second factor was the exposure to the mixtures or the viability evaluation time with eight levels (0.0, 0.5, 1.5, 3.0, 6.0, 12.0, and 25.0 hours). The results were compared with the Rhapsody® biological control and two replicates were used per treatment.

The evaluated response variable was the number of CFU/mL in each of the evaluation times. Additionally, too was determined, corresponding to the time when 50% of the biomass of B. subtilis EA-CB0015 loses its viability due to the exposure to each of the mixtures, using an univariate design for data analysis, wherein the factor is the percentage (%) of cell death.

The percentage of cell death after three and twenty-five hours was also determined, (3 hours is the average time it takes to apply a composition after it has been prepared and 25 hours is the maximum time that a composition remains in the mixing tanks before being applied).

Table 7 shows t_{d50} (the time when 50% of the B. subtilis EA-CB0015 spores lose viability) and the percentage of cell death after 3 and 25 hours for the composition of B. subtilis EA-CB0015 in each fungicide mixture. For its production, the culture was taken to a stirred tank and mixed with the respective composition adjuvants. M1, M2, M3, M4, M5, M6, M7, and M8 denote the various fungicide mixtures to which the formulations were subjected.

TABLE 7
Cell death percentage and t_{d50} of B. subtilis EA-CB0015
after mixing with various chemical fungicide mixtures.

| | t_{d50} MH₂O | % cell death after | % cell death after |
|---|---|---|---|
| Mixture | (hours) | 3 hours | 25 hours |
| M1 | >25 | 3.7 | 24.6 |
| M2 | >25 | 18.6 | 10.6 |
| M3 | >25 | 32.1 | 25.4 |
| M4 | >25 | 25.9 | 22.0 |
| M5 | >25 | 16.3 | 36.4 |
| M6 | >25 | 7.2 | 28.1 |
| M7 | >25 | 17.0 | 17.4 |
| M8 | >25 | 6.0 | 6.5 |

The above table indicates that the composition of B. subtilis EA-CB0015 showed a too greater than 25 hours for all mixtures, reaching average viability reductions of only 20.1%. Furthermore, given that viability reductions in the composition were lower than 50%, the composition of the present invention most likely will provide extra protection to B. subtilis EA-CB0015, allowing to maintain viability for long periods of exposure to fungicide mixtures.

Regarding the percentage of viability loss, it was observed that in ordinary conditions, (3 hours) M2 (SICO®), M3 (Siganex®), and M4 (Baycor®) mixtures showed the highest bacterial viability reduction rates in the composition, whereas in extraordinary conditions (25 hours), M1 (Bravonil®), M3 (Siganex®), M5 (Bumper®), and M6 (Opus®) showed the highest reduction rates.

### Example 9

### Evaluating the Effect of the Compositions of the Invention on the Severity of Black Sigatoka. Greenhouse and Field Tests

An evaluation of the effect of the bacterial water-based composition of B. subtilis EA-CB0015 on Black Sigatoka in greenhouse conditions was performed. To this end, 4-month old c.v. Williams banana plants were used and pathogen inoculation was made artificially by applying a mycelial suspension of the M. fijiensis fungus to leaf number one of the plant. The ascospore discharge methodology used by Cenibanano was employed to obtain the mycelium of M. fijiensis for the inoculation of the plants [11].

The compositions were applied one day after the inoculation of the plants with the pathogen. The compositions were diluted to a concentration of 1.0×10⁸±0.1 CFU/mL and applied using a Mini Spray gun with cup K-3® airbrush with fan sprayer connected to a 30-psi compressor and calibrated for spraying 50 drops/cm² at a distance of 30 cm. The top and underside of the infected leaves were fumigated only once at a distance of 30 cm, ensuring a minimum concentration of 50 drops/cm².

A single-factor design was used for this experiment to evaluate the water-based composition (MH₂O y P3), a chemical control: Bravonil® in water, a biological control: Serenade® (1×10⁸±0.1 CFU/ml), and sterile water as absolute control.

Disease development was measured one month after the plants were infected. The degree of infection was determined using the Fouré scale (1982) [17] and the necrotic area was determined using a 8 Mega-pixel Samsung camera and Zeiss®'s Axio Visio® 4.2 image processing software.

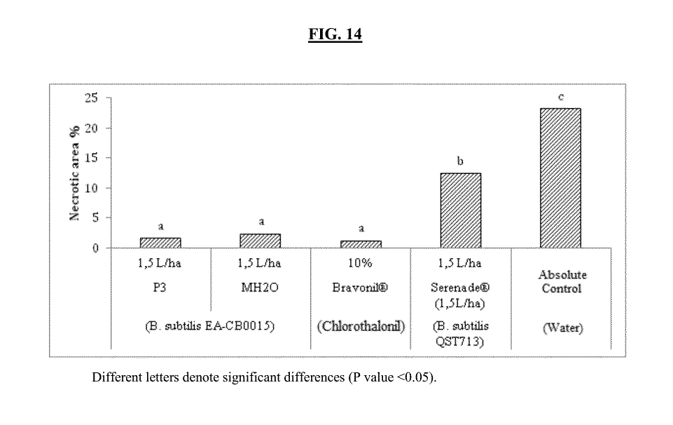

The analysis of variance (ANOVA) indicated that there are statistically significant differences among the treatments. To determine these differences, a multiple range test was conducted using the Turkey method. The percentages of necrotic area for the various treatments are shown in FIG. 14.

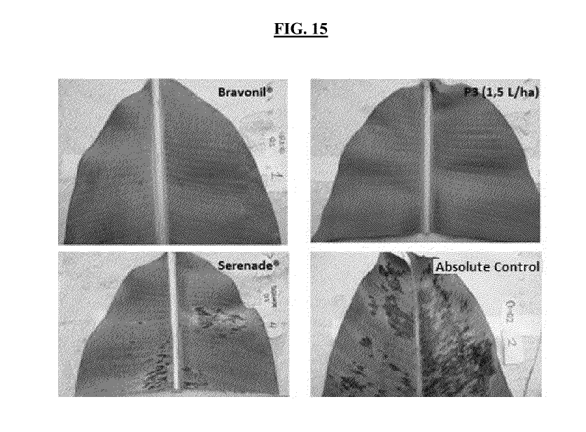

As shown in FIG. 14, the water-based mixture composition (MH2O and P3) of B. subtilis EA-CB0015 according to the present invention and the Bravonil 720® positive control significantly reduced the percentage of necrotic area of banana leaves with percentages of 1.67% and 1.26%, respectively. FIG. 15 includes photographs of leaves subjected to each of the treatments, which visually show the difference in appearance of post-treatment leaves. The photographs were selected from portions of average leaves.

To determine the effectiveness of the water-based mixture composition of B. subtilis EA-CB0015, the product was evaluated at field level in a lot of 1.5 ha, with three plots per treatment. Each plot of 220 m² contained 42 plants; six central plants were taken per plot in order to evaluate the disease. Treatments were applied every 11 days with a motor sprayer (Stihl® SR-420) of 15 L capacity, spraying 60 drops per cm² of leaf.

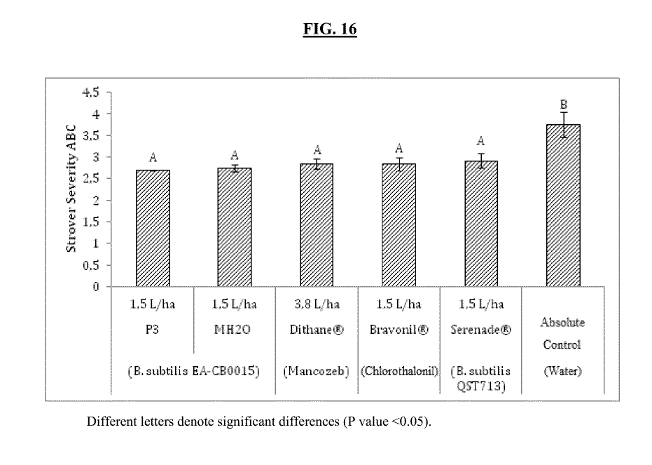

The evaluation of Black Sigatoka disease was performed using two methodologies: biological warning and severity by Stover. FIG. 16 shows the area under the curve (AUC) for the severity of Black Sigatoka obtained during 14 weeks of evaluation. This figure shows that both water-based compositions of B. subtilis EA-CB0015 (MH₂O y P3) reduced disease severity with no significant differences when compared with chemical controls (Bravonil®, Dithane®) and biological control (Serenade®).

### Example 10

### Effect of B. subtilis EA-CB0015 on Botrytis cinerea in Pompoms

The effect of B. subtilis EA-CB0015 on Botrytis Cinerea in pom poms was evaluated. The pompoms were disinfected for 1 min in sodium hypochlorite 1%, washed with sterile distilled water, and finally allowed to dry. Then, each flower was placed in a disposable cup and each treatment was sprayed with an airbrush. After 24 hours of applying the treatments, the pathogen (B. cinerea) at a concentration of 5*10̂3 spores/mL using an atomizer (2 mL) was applied and incubated at an average temperature of 20° C. and a relative humidity above 90%.

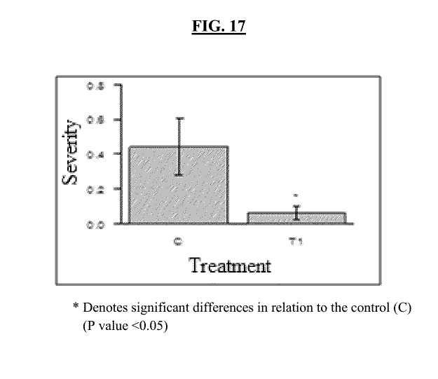

Disease measurement was performed after 7 days according to the percentage of affected petals and the severity. FIG. 17 shows that the spore suspension of B. subtilis EA-CB0015 (T1) decreased the severity of the disease by 84% when compared with the untreated control (C).

### Example 11

### Effect of B. subtilis EA-CB0015 on Colletotrichum sp. in Tree Tomato (Cvphomandra betacea)

The effect of B. subtilis EA-CB0015 on Colletotrichum sp. in tomato tree was evaluated. To this end, tomatoes were disinfected for 2 minutes in 70% ethanol, washed with sterile distilled water, and finally allowed to dry.

Then, a puncture of less than 2 mm in depth was made in the halfway region of the fruit and 25 μL of water (C) or spores of B. subtilis EA-CB0015 (T1, concentration 1*10⁷ CFU/mL) were applied. After 24 hours, the puncture was inoculated with 15 μL of Colletotrichum sp. EAHP-007 at a concentration of 400,000 spores/mL.

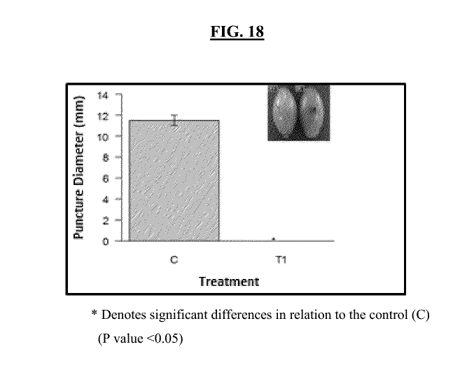

FIG. 18 shows the effects of the suspension of spores of B. subtilis EA-CB0015 on the diameter of the puncture generated by Colletotrichum sp. EAHP-007 in the fruit, achieving effective control of the disease.

### REFERENCES

[1] M. Shoda, <<Bacterial Control of Plant Disease,>> Journal of Bioscience and Bioengineering, pp. 515-521, 200.
[2] H. P. Bais, R. Fall and J. M. Vivanco, <<Biocontrol of Bacillus subtilis against infection of Arabidopsis roots by Pseudomonas syringae is facilitated by biofilm formation and surfactin production,>> Plant Physiology, vol. 134, pp. 307-319, 2004.
[3] T. Stein, <<Bacillus subtilis antibiotics: structures, syntheses and specific functions,>>Molecular Microbiology, vol. 56, pp. 854-857, 2005.
[4] M. Ongena and P. Jacques, <<Bacillus lipopeptides: versatile weapons for plant disease biocontrol,>>Applied Microbiology and Biotechnology, vol. 16, No. 3, pp. 115-125, 2008.
[5] G. Winkelmann, R. Allgaier and G. Jung, <<Iturin AL A New Chain Iturin A possessing an unusual high content of C16-β-amino acids,>>Journal of Antibiotics, vol. 16, pp. 1451-1457, 1983.
[6] N. Vanittanakon y W. Loeffler, <<Fengycin-a novel antifungal lipopeptide antibiotic produced by Bacillus subtilis F-29-3,>> n° 7, pp. 888-901, 1986.
[7] F. Peypoux, J. M. Bonmatin and J. Wallach, <<Recent trends in the biochemistry of surfactin,>> vol. 51, pp. 553-563, 1999.
[8] Y. H. Wei, L. F. Wang, J. S. Changy and S. S. Kung, <<Identification of induced acidification in in iron-enriched cultures of Bacillus subtilis during biosurfactant fermentation,>> Journal of Bioscience and Bioengineering, vol. 96, No. 2, pp. 174-178, 2003.
[9] X. J. Tang, G. Q. He, Q. H. Chen, X. Y. Zhang and M. Ali, <<Medium optimization for the production of thermal stable beta-glucanase by Bacillus subtilis ZJF-1A5 using response surface methodology,>> Bioresource Technology, vol. 93, No. 2, pp. 175-181, 2004.
[10] J. Pelaez, L. E. Vasquez, T. J. Diaz, D. A. Castañeda, E. Rodriguez and R. E. Arango, <<Use of a micro title plate dilution assay to measure activity of antifungal compounds against Mycosphaerella fijiensis Morelet, Revista Facultad Nacional de Agronomia, vol. 59, n° 2, pp. 4325-3433, 2006.
[11] Dupont, <<Técnicas mejoradas para el manejo e identificaciÓn,>> Boletin Técnico E55786, 1982.
[12] A. G. C. I. and G. A. Riveros, <<Microbiological control of black leaf streak disease,>> de Mycosphaerella leaf spot disease of bananas: present status and outlook, Montpellier, INIBAP, 2003, pp. 287-295.
[13] S. Talavera, E. Bustamante, R. González and V. Sánchez, <<SelecciÓn y evaluaciÓn en laboratorio y campo de microorganismos glucanolíticos antagonistas a Mycosphaerella fijiensis,>> Manejo Integrado de Plagas, vol. 47, pp. 24-30, 1998.
[14] P. Jacques, C. Hbid, J. Destain, H. Razafindralambo, M. Paquot, E. De Pauw and P. Thonart, <<Optimization of biosurfactant lipopeptide production from Bacillus subtilis S499 by Plackett-Burman Design,>> Applied Biochemistry and Biotechnology, Vols. %1 de %277-79, pp. 223-233, 1999.
[15] S. K. Brar, M. Verma, R. D. Tyagy, J. R. Valero and R. Y. Surampalli, <<Screening of different Adjuvants for Waste/water sludge-based Bacillus thuringiensis formulation,>> Microbial Control, vol. 99, n° 4, pp. 1065-1079, 2006.
[16] H. Burges, Formulation of microbial biopesticidas, beneficial microorganisms, nematodes and seed treatments, USA: Kluwer Academic Publishers, 1998.
[17] E. Foure, <<Les cercosporioses du bananier et leurs traitements. Comportement des varietés. Etude de la sensibilite varietale des bananiers et plantains e Mycosphawerella fijiensis Morelet au Gabomn (maladie desreies noires). I-Incubation et evolution de la maladie,>>Fruits, vol. 37, pp. 749-771, 1982.
[18] G. Holz, S. Coertze and B. Williamson, <<The Ecology of Botrytis on Plant Surfaces,>> de Botrytis: Biology, Pathology and Control, Netherlands, Springer, 2007, pp. 9-27.
[19] AgraQuest, <<Serenade,>> 2009. [Online]. Available at: www.agraquest.com. [Last accessed on: 17 03 2010].
[20] P. Tudzynski and L. Kokkelink, <<). Botrytis cinerea: Molecular Aspects of a Necrotrophic Life Style,>> de Plant Relationships, Berlin, Springer, 2009, pp. 29-50.

It should be understood that the present invention is not limited to the embodiments described and illustrated herein. As it will be apparent to one skilled in the art, there are potential variations and modifications that do not depart from the spirit of the invention, which is only defined by the following claims:

1) A procedure for increasing the production of biomass and metabolites of microorganisms of Bacillus sp. species, comprising the culture of the microorganism in a suitable culture medium under specific environmental conditions.

2) A procedure according to claim 1, wherein the microorganism is selected from the group consisting of Bacillus subtilis and Bacillus amyloliquefaciens.

3) A procedure according to claim 1, wherein the microorganism is Bacillus subtilis EA-CB0015 or Bacillus amyloliquefaciens EA-CB0959.

4) A procedure according to claim 1, wherein the suitable culture medium comprises one or more components selected from the group consisting of carbohydrates, yeast extract, ammonium sulfate, peptone, salts containing magnesium, sulfur, manganese, chlorine, potassium, phosphorus, calcium, and sodium either in a solid, semisolid, or liquid matrix.

5) A procedure according to claim 4, wherein the suitable culture medium has the following composition:

| COMPONENT | Concentration (g/L) |
|---|---|
| Glucose | 30.0-35.0 |
| Yeast extract | 30.0-35.0 |
| MnSO₄ | 0.025-0.05 |
| Calcium chloride | 0.02-0.04 |
| Ammonium sulfate | 0.80-1.20 |
| MgSO₄ | 3.50-5.00 |
| HPO₄ | 0.40-0.60 |
| KH₂PO₄ | 0.40-0.60 |

6) A procedure according to claim 1, wherein the specific environmental conditions include pH, temperature, stirring speed, fermentation time, and aeration.

7) A procedure according to claim 6, wherein the specific environmental conditions of culture are:

| Stirring speed | 400-600 | rpm |
|---|---|---|
| Aeration | 1-5 | vvm |
| pH | 5.5-7.5 | |
| Temperature | 20-40° | C. |
| Fermentation time | 10-100 | hours |

8) A procedure according to claim 1, wherein the obtained biomass is additionally separated by centrifugation and/or microfiltration.

9) A procedure according to claim 1, wherein the metabolites are additionally extracted by solvent extraction, precipitation, adsorption, or chromatography.

10) Biomass of Bacillus subtilis or Bacillus amyloliquefaciens obtained by a procedure according to claims 1 to 8.

11) Biomass of Bacillus subtilis EA-CB0015 obtained by a procedure according to claims 1 to 8.

12) Biomass of Bacillus amyloliquefaciens EA-CB0959 obtained by a procedure according to claims 1 to 8.

13) Metabolites of Bacillus subtilis and/or metabolites of Bacillus amyloliquefaciens, obtained by a procedure according to claims 1 to 9.

14) Metabolites of Bacillus subtilis EA-CB0015 and/or metabolites of Bacillus amyloliquefaciens EA-CB0959, obtained by a procedure according to claims 1 to 9.

15) Metabolites of Bacillus subtilis EA-CB0015 and/or Bacillus amyloliquefaciens EA-CB0959 according to claim 14, characterized by being lipopeptides of the surfactin, iturin, and fengycin families.

16) bolites of Bacillus subtilis EA-CB0015 according to claim 15, wherein the lipopeptides of the fengycin family correspond to fengycin C having the general formula:
R-Glu1-Orn2-Tyr3-Thr4-Glu5-Va16-Pro7-Gln8-Thr9-Ile10
wherein R corresponds to a saturated or unsaturated hydrocarbon chain of 14 to 18 carbons.

17) Metabolites of Bacillus subtilis EA-CB0015 and/or metabolites of Bacillus amyloliquefaciens EA-CB0959 according to claims 13 to 16, with antimicrobial activity.

18) A composition comprising biomass of Bacillus subtilis EA-CB0015 according to claim 11 and/or metabolites thereof, together with an agrochemically acceptable carrier.

19) A composition comprising biomass of Bacillus amyloliquefaciens EA-CB0959 according to claim 12 and/or its metabolites, together with an agrochemically acceptable carrier.

20) A composition comprising metabolites of Bacillus subtilis EA-CB0015 and/or metabolites of Bacillus amyloliquefaciens EA-CB0959 according to claim 14, together with an agrochemically acceptable carrier.

21) A composition according to any of claims 18 to 20, further comprising one or more biocidal agents selected from the group consisting of anilinopyrimidines, bitartenols, sterols, difeconazole, tebuconazole, epoxiconazole, mancozeb, and cloratolonil.

22) A composition according to any of claims 18 to 21, comprising:

| COMPONENT | CONCENTRATION | |
|---|---|---|
| Culture of Bacillus sp. | 86.6%-93.2% | v/v |
| at a concentration of | | |
| 7.0 to 18.0 g/L | | |
| Sodium carboxymethyl cellulose | 2.0-4.0 | w/v |
| phosphate buffer 3M pH 5 | 1.0-5.0 | v/v |
| Glycerol | 1.0-4.0 | v/v |
| Tween 20 | 0.25-0.75 | v/v |
| Triton X100 | 0.25-0.75 | v/v |
| Potassium sorbate | 0.01-0.1 | v/v |
| Xanthan gum | 0.05-0.15 | w/v |
| Skimmed milk | 0.20-1.50 | w/v |
| Titanium dioxide | 0.03-1.00 | w/v |

23) Use of biomass of Bacillus subtilis EA-CB0015 and/or biomass of Bacillus amyloliquefaciens EA-CB0959 according to claims 11 and 12 for controlling plant pathogens such as Mycosphaerella fijiensis, Fusarium oxysporum, Ralstonia solanacearum, Botrytis cinerea, Colletotrichum sp., Monilia sp., Rhizoctonia solani and Fusarium solani.

24) Use of biomass of Bacillus subtilis EA-CB0015 and/or Bacillus amyloliquefaciens EA-CB0959 according to claim 14 for controlling plant pathogens such as Mycosphaerella fijiensis, Fusarium oxysporum, Ralstonia solanacearum, Botrytis cinerea, Colletotrichum sp., Monilia sp. Rhizoctonia solani and Fusarium solani.

25) Use of a composition according to claims 18 to 22 for controlling plant pathogens such as Mycosphaerella fijiensis, Fusarium oxysporum, Ralstonia solanacearum, Botrytis cinerea, Colletotrichum sp., Monilia sp., Rhizoctonia solani and Fusarium solani.

26) A method for the treatment of crops against phytopathogenic agents that comprises the application of an effective biomass amount of Bacillus subtilis EA-CB0015 and/or biomass of Bacillus amyloliquefaciens EA-CB0959 according to claims 11 and 12.

27) A method for the treatment of crops against phytopathogenic agents that comprises the application of an effective amount of metabolites of Bacillus subtilis EA-CB0015 and/or Bacillus amyloliquefaciens EA-CB0959 according to claim 14.

28) A method for the treatment of crops against phytopathogenic agents such as Mycosphaerella fijiensis, Fusarium oxysporum, Ralstonia solanacearum, Botrytis cinerea, Colletotrichum sp., Monilia sp., Rhizoctonia solani y Fusarium solani, comprising the application of an effective amount of a composition according to claims 18 to 22.
